(19) United States
(12) Patent Application Publication
(10) Pub. No.: US 2003/0165584 A1
(43) Pub. Date: Sep. 4, 2003

(54) Agent for regulating lipidic metabolism and method for producing said agent

(76) Inventor: Sergey Petrovich Semenov, Sankt Petersburg (RU)

Correspondence Address: HENRY M FEIEREISEN, LLC, 350 FIFTH AVENUE, SUITE 4714, NEW YORK, NY, 10118, US

(21) Appl. No.: 10/362,620

(22) PCT Filed: Aug. 22, 2001
(86) PCT No.: PCT/RU01/00357

(30) Foreign Application Priority Data
Aug. 23, 2000 (RU) 2000122087
Apr. 12, 2001 (RU) 2001109741

(51) Int. Cl. A61K 35/78

(52) U.S. Cl. 424/750

(57) ABSTRACT

The invention is directed on creation of agent of a vegetative origin rendering the medical—preventive influence on alive organism and intend for prophylactic of atherosclerosis and decrease the weight of a body and reduction of a level of cholesterol in whey of blood.

The agent for regulating of lipidic metabolism represents the extract received by processing non-polar extractant of crushed grains of millet or not crushed grains of oats.

The agent received from grains of millet includes lipid fraction with density at 20° C.-0.917-0.925 g/cm³, acid number 120.0-145.0 mg KOH/g, iodine number 87.0-93,0 and contains more than 80% of free greasy acids.

The agent from grains of oats includes lipid fraction with density at 20° C.-0.760-0.925 g/cm³, acid number—103.0-110.0 mg KOH/g, iodine number—132,0-140, thus lipid fraction contains 57-62% of free greasy acids and 27-32% and 25-29% of ethers of free greasy acids.

The method for producing of a agent for regulating of lipidic metabolism is characterized by that the crushed grains of millet or the not crushed grains of oats process by non-polar extractant, chosen from group: petroleum ether, hexane, diethyl ether, chloroform, at—ratio raw material—extractant 1:2-1:4 and temperature (40-70)° C. with the subsequent separation and concentration of a target product.

The received agent from grains of millet or grains of oats expands the assortment of agent intended for medical—preventive influence on alive organism and having antisclerotic activity.

### FIELD OF THE INVENTION

The invention concerns to area of medicine, is concrete to elaboration of means of a vegetative origin rendering medical preventive influence on alive organism and is intended for prophylactic of atherosclerosis and decrease the level of cholesterol in whey of blood.

### BACKGROUND OF THE INVENTION

There are various means necessary for ability to a man's life, known as sources of irreplaceable greasy acids. Among them the special place is occupied by vegetable oils, for example sunflower, corn, rape, mustard, containing linoleic acid, that positively works in atherosclerosis, reducing a level of cholesterol, arterial pressure and etc.

There is a method of reception of a medical product from grain cultures including wetting of raw material in water, growing of grains, drying and crushing of the dried up grain (RU the U.S. Pat. No. 2,137,400, A 23 L 1/30).

Medical preparation having a prolong action of biologically active substances being received from extracts of medicinal plants, immobilized by the account of adsorption on grains of cereal cultures (RU the U.S. Pat. No. 2,111,765, A 23 D 9/00,1998).

The extracts of medicinal plants fill in a vessel containing grains of cereal cultures in the ratio 1:0,5. Immobilization is carrying out within 24-36 hours then the grains are drying during 3-5 hours at temperature 55-60° C.

The most active medical influence on organism of the man, renders the linen oil, which contains linolenic, linoleic, oleic and sated greasy acids in the ratio 6-8:0.5-3:0.6-3:0.4-3 (RU the U.S. Pat. No. 2,158,514, A 23 D 9/00, 2000).

The known method of manufacture of medical—preventive product from vegetative and/or of animal raw material consists in its preparation, extraction, preparation of an extract biologically of active substances and mixing of prescription ingredient for preparation of a composition with subsequent concentration up to humidity no more than 22%.

As vegetative raw material is used medicinal plants and/or their compositions, and as a sweetener—honey (RU, U.S. Pat. No. 2,137,400, A 23 L 1/30, 1/305,1/308).

There is a method for producing of a medical product from wheat including wetting of raw material in water, growing of grains, drying and crushing of the dried up grain to the size of particles 20-30 Mk (RU, U.S. Pat. No. 2,137,400, And 23 L 1/30, 1/305, 1/308).

### SUMMARY OF THE INVENTION

The invention is directed on the creation of the agent regulating the lipidic metabolism of organism and also on the development of the method of its reception.

The achievable technical result consists in expanding of assortment of means rendering medical—preventive influence on alive organism and having antisclerotic activity.

The method Ifor regulating of lipidic metabolism represents the extract received by processing with an non-polar extractant of crushed grains of millet or of not crushed grains of oats.

The agent received from grains of millet includes the lipid fraction with density at 20° C.-0.917-0.925 g/cm³, acid number 120.0-145.0 mg KOH/g, iodine number 87.0-93.0 and contains more than 80% of free greasy acids.

The agent from grains of oats includes the lipid fraction with density at 20° C.-0.760-0.925 g/cm³, acid number—103, 0-110,0 mg KOH/g, iodine number—132,0-140, thus the lipid fraction contains 57-62% of free greasy acids and 27-32% and 25-29% of ethers of free greasy acids.

The method for producing of agent I for regulating of lipidic metabolism is characterized by that the crushed grains of millet or the not crushed grains of oats process with non-polar extractant at their ratio 1:2-1:4 and temperature (40-70)° C. with the subsequent separation and concentration of a target product.

As an non-polar extractant use the solvent chosen from group: petroleum ether, hexane, diethyl ether, chloroform.

The extraction of millet grains carry out within not less than 3 hours, and the extraction of oats grains—within 6-12 hours, a target product separate by a filtration, thus a target product concentrate by evaporation.

Grains of millet crush up to the size of particles of 0.2-0,5 mm.

The received extract of the crushed grains of millet represents lipid fraction of millet grains described on the chemical composition more than 80% of free greasy acids and less than 20% triglycerides.

Chromatographic analysis of lipid fractions received from grains of oats and millet, is made up on an instrument complex of Schimadzy's firm (Japan) including gas chromatograph GC-17A, mass spectrometer QP5000 and system of data processing, a column capillary of melted quartz SPB-5 (Supeico) of length 25 m and internal diameter 0.2 mm. Adjustment of mass spectrometer carried out in an automatic mode on perfluorotributylamine.

Dosing sample (0.2 mkl) was carried out in a mode split. Temperature of a column was programmed from 40° C. (5 min.) up to 270° C. (20 min.) with speed 10 grades/min.

Chromatographic researches have shown that the product received from grains of millet has the following chemical composition given in table 1.

TABLE 1

| No. | Name of compound | Contents in sample, % |
|---|---|---|
| 1 | Linoleic acid | 60-70 |
| 2 | Palmitic acid | 5-7 |
| 3 | Methyl ether of linoleic acid | 2-4 |
| 4 | Methyl ether of oktadecanoic acid | 1.5-3 |
| 5 | Not sated hydrocarbons | 3.5-4.5 |
| 6 | Not sated oxygen-containing | Rest |
| | aldehydes and compounds of greasy acids | |

The product received from grains of oats has the following chemical composition given in table 2.

TABLE 2

| No. | Name of compound | Contents in sample, % |
|---|---|---|
| 1 | Linoleic acid (9,12-octadecandiene acid) | 37-40 |
| 2 | Methyl ether of linoleic acid | 22-25 |
| 3 | Palmitic acid (hexadecanoic acid) | 20-22 |
| 4 | Isopropylpalmitate | 5-7 |
| 5 | Octadecyl spirit | 4 |
| 6 | Greasy spirit | 0.8 |
| 7 | Myristic acid (tetradecanoic) | 0.4 |
| 8 | Ethylmyristat | 0.1 |
| 9 | Isopropyl myristat | 0.03 |
| 10 | Not identified alkenes | Rest |

The received agent is intended for prophylaxis of atherosclerosis and decrease of weight of a body at the expense of stimulation of lipolytic activity of whey blood that is accompanied by decrease in it's concentration of triglycerides and cholesterol.

The researches have shown that the application of the agent not only stimulates activity of lipase in organism but also creates simultaneously conditions for the amplified oxidation of greasy acids with their primary use as a source of power supply.

### EXAMPLE 1

Grains of millet with humidity 12.8% crush up to the size of particles 0.2-0.5 mm and extract by petroleum ether at temperature 60° C. at a ratio raw material—extracting agent 1:4. Extraction was carried out in the apparatus Soxlet, time of extraction—6 hours. The received extract filter and evaporate before complete removal of extracting agent. The received extract represents lipid fraction of grains of millet.

The output of lipid fraction from absolute dry raw material has made—2.4%.

The characteristic of an extract:

| Color | yellow with an orange shade |
|---|---|
| Transparency | transparent |
| Density at 20° C. | 0.917 g/cm³ |
| Parameter of refraction at 20° C. | 1.467 |
| Acid number, mg KOH on 1 g | 137.0 |
| Iodine number (on Kaufman) | 89.0 |

Rats—male of a line Vistar (Rappolovo), weight 190-210 g. were used in the experiment. The animals received the lipid fraction of grains of millet in a doze 0.3 ml 12-multiply daily till 5 times per week with the help of a gastric sound. After the period of injection of a agent the rates decapitated, collected blood and after formation of a clot separated whey by centrifuging. In the received whey defined the contents of free greasy acids on reaction of formation of a copper complex giving with diethyl carbamate colouring, which intensity measured on spectrophotometer Cφ-46 at length of a wave 440 nm. Activity of lipase of whey estimated by turbodimetric method. The contents of triglycerides and general cholesterol defined on the analyzer Spectrum by fermentative method. Phenotype of lipids of whey was carried out on the basis of the analysis of their fraction structure.

Date about biochemical index of rates whey are given in table 3.

TABLE 3

| | Control index | |
|---|---|---|
| | (Without injection of | Experience index |
| The name of a index | a agent) | (12-multiple injection) |
| Activity lipase, % | 13.2 ± 1.3 | 27.8 ± 2.0 |
| Cholesterol, mmol, % | 1.7 ± 0.1 | 1.2 ± 0.1 |
| Triglycerides, mmol/lit. | 0.96 ± 0.1 | 0.67 ± 0.08 |
| Lipoproteides of high | 0.65 ± 0.08 | 0.56 ± 0.09 |
| density, mmol/lit. | | |
| Lipoproteides of low | 0.54 ± 0.07 | 0.61 ± 0.1 |
| density, mmol/lit | | |
| Lipoproteides of very | 0.46 ± 0.02 | 0.29 ± 0.01 |
| low density, mmol/lit. | | |
| Atherogtnous factor | 1.53 ± 0.1 | 1.55 ± 0.1 |
| Free greasy acids, | 291.6 ± 32.5 | 273.12 ± 25.2 |
| mkmol/lit. | | |

As follows from the given data the activity of lipase of whey of blood by using the lipid fraction of grains of millet grew more than in 2 times. The contents of general cholesterol and triglycerides at these animals were reduced to 70% and 69.7% on the average accordingly. The injection of a agent did not result in change of size of factor of atherogtnous and contents of lipoproteides of various classes. The contents of lipoproteides of very low density has decreased up to 63%.

The received data testify that the application of lipid fraction of grains of millet stimulates lipolytic activity of whey of blood that is accompanied by decrease in it concentration of cholesterol. The injection of the agent makes active the lipase and creates conditions for the amplified oxidation of greasy acids with their primary use as a source of power supply.

The longer injection of the agent (24-multiple) resulted in decrease of concentration peratherogenous lipoproteides of low density and decrease the factor of atherogenous in 1.8 times. Besides the content of free greasy acids with 279.4 up to 253.0 mmol/lit. reduced. These changes convincingly demonstrate antiatherogenous efficiency of the agent at rather long it's application and decrease risk of progress of atherosclerosis.

The data on change of rats body weight having received the lipid fraction of grains of millet are given in table 4.

TABLE 4

| | Control index | | Experimental index | |
|---|---|---|---|---|
| Duration of | Prior to the | | Prior to the | After the |
| injection of | beginning of | After the end | beginning of | end of |
| the agent | experience | of experience | experience | experience |
| 12-multiple | 192 ± 4.1 | 22.4 ± 2.4 | 203.0 ± | 214.0 ± |
| injection | | | 5.7 | 6.4 |
| GAIN | 16.7 | | 5.4 | |
| 24-multiple | 212.0 ± 16.8 | 280.0 ± 16.1 | 199.0 ± | 229.0 ± |
| injection | | | 6.1 | 5.7 |
| GAIN | 32.1 | | 15.1 | |

The given data testify that 12-multiple receptions of the agent brake a gain of weight of a body in 3 times and 24-multiple on the average—in 2 times.

### EXAMPLE 2

Grains of oats by humidity 7.0% extract by petroleum ether at temperature 60° C. at ratio extractant: raw material 4:1. Extraction was carried out in the device Soxleta, time of extraction 6 hours. The received extract is filtered and evaporated before complete removal of extracting agent. The received extract represents the lipid fraction of grains of oats.

The characteristic of an extract:

| Color | olive |
|---|---|
| Transparency | transparent |
| Density at 20° C. | 0.925 g/cm³ |
| Parameter of refraction at 20° C. | 1.475 |
| Acid number (mg KOH on 1 g) | 103.0 |
| Iodine number | 109 |
| Saponification number | 171 |

The influence of lipid fraction on index of a condition of metabolism of lipids at experimental animals was studied.

In experiment were used rats—male of a line Vistar (Rappolovo) in weight 160-200 g. The lipid fraction was entered to rats in a doze 0.3 ml with the help of a gastric probe 12-multiply daily till 5 times in one week. After the period of injection of a lipid fraction, rats were decapitated, collected blood and after formation of a clot separated whey by centrifuging.

In the received whey defined the contents of free greasy acids on reaction of formation of a copper complex giving with diethyl carbamate colouring.

Activity of lipase of whey estimated by turbodimetric method.

The contents of triglycerides and general cholesterol defined on the analyzer Spectrum (ABBOT, USA) by fermentatives methods. Phenotype of lipids of whey was carried out on the basis of the analysis of it's fraction structure.

On the data of biochemical and morphological researches the lipid fraction does not render on damaging influence on parenchyma of internal bodies and is characterized by low allergen activity.

Date about biochemical index of rats whey are given in table 5.

TABLE 5

| Index | Control | Experience |
|---|---|---|
| Lipase, unit/lit. | 12.0 ± 4.3 | 20.3 ± 3.8 |
| Cholesterol, mmol/lit | 2.2 ± 0.1 | 2.1 ± 0.2 |
| Triglycerides, mmol/lit | 0.99 ± 0.32 | 1.0 ± 0.24 |
| Lipoproteides of high density, mmol/lit | 0.86 ± 0.11 | 1.25 ± 0.30 |
| Lipoproteides of low density, mmol/lit | 0.91 ± 0.21 | 0.40 ± 0.15 |
| Lipoproteides of very low density, | 0.42 ± 0.16 | 0.45 ± 0.11 |
| mmol/lit | | |
| Atherogtnous factor | 1.55 ± 0.25 | 0.76 ± 0.14 |
| The correlation of cholesterol/LPHD | 2.78 ± 0.45 | 1.67 ± 0.21 |
| Free greasy acids, mkmol/lit | 312.4 ± 38.6 | 289.6 ± 32.1 |

The activity of lipase of rats blood whey received the lipid fraction that follows from the given data grew by 60%, contents of lipoproteides of high density—on 45% and contents of lipoproteides of low density was reduced on 56% on the average.

The size of atherogenous factor and correlation of the contents of general cholesterol whey to the contents of lipoproteides of high density as a result of introduction of lipid fraction has decreased in 2 times and on

40% accordingly.

Lipid fraction has antiatherogenous potential, which is shown in increase of the contents in whey of blood of lipoproteides of high density, having antiatherogenous potential and—in decrease peratherogenous lipoproteides of low density.

The size of atherogenous factor was essentially reduced concerning the general contents of cholesterol to the contents of high density of lipoproteides.

The significant increase the activity of lipase in the whey is not accompanied by increase of concentration of free greasy acids. It is probably connected that lipid fraction stimulates them utilization with primary use as power of substrates.

The application of lipid extract though does not cause decrease of cholesterol and triglycerides in whey, however, brings the essential changes in lipoproteides phenotype causing its shift on the benefit of antiatherogenesis.

Thus, the developed agent can be used for prophylactic of atherosclerosis.

The data on dynamics of weight of a body of experimental animals are submitted in table 6.

TABLE 6

Weight of a body in grams

| Group of animals | Before injection | After injection |
|---|---|---|
| THE CONTROL | 170 ± 6 | 192 ± 9 |
| EXPERIENCE | 170 ± 6 | 178 ± 7 |

The given data testify that the daily introduction of an extract inhibited in gaining the weight of a body during the period of supervision.

The long introduction of lipid extract does not result in changes of the contents of hemoglobin and erythrocyte in peripheral blood of rats. Research of structure of formal elements at this animal showed the absence of infringement of leucocyte formula in comparison with the control.

Thus, the long introduction of the agent does not render damaging influence on blood formation system of animals.

The agent is characterized by low allergen activity.

### Industrial Applicability

The received agent from grains of millet or grains of oats expands the assortment of agent intended for medical—preventive influence on alive organism and having an antisclerotic activity.

The researches have shown that lipid fractions from grains of oats or millet can be used for prophylactic of atherosclerosis and decrease the weight of a body.

What is claimed is:—

1. Agent for regulating of lipidic metabolism is characterized that it represents the extract received by processing of crushed grains of millet or not crushed grains of oats by non-polar extractant, thus the agent from grains of millet includes the lipid fraction with density at
20° C. 0.760-0.925 g/cm³, acid number 120,0-145.0 mg KOH/g, iodine number 87.0-93.0 and contains more than 80% of free greasy acids, and the agent from grains of oats includes the lipid fraction with density at 20° C.-0.760-0.925 g /cm³, acid number—103.0 mg KOH/g, iodine number—109, thus the lipid fraction contains 57-62% of free greasy acids and 25-29% of ethers of free greasy acids.

2. The agent of claim 1, is characterized that it is intended for prophylactic of atherosclerosis.

3. The agent of claim 1, is characterized that it is intended for decrease of weight of a body.

4. Method for producing of a agent for regulating of lipidic metabolism is characterized that the crushed grains of millet or not crushed grains of oats process by non-polar extractant at a ratio raw material—extractant 1:2-1:4 and temperature (40-70)° C. with the subsequent separation and concentration of a target product.

5. The method of claim 4, is characterized that as non-polar extractant use the solvent chosen from group: petroleum ether, hexane, diethyl ether, chloroform.

6. The method of claim 4, is characterized that grains of millet is crushing up to the size of particles of 0.2-0.5 mm.

7. The method of claim 4, is characterized that extraction of grains of millet is carrying out within not less 3 hours.

8. The method of claim 4, is characterized that extraction of grains of oats is carrying out within 6-12 hours.

9. The method of claim 4, is characterized that a target product separate by filtration.

10. The method of claim 4, is characterized that a target product concentrate by evaporation.
